(19) United States
(12) Patent Application Publication
(10) Pub. No.: US 2008/0014601 A1
(43) Pub. Date: Jan. 17, 2008

(54) CONTROLLER FOR A BLOOD PARAMETER TESTING SYSTEM AND METHOD OF TESTING THEREFOR

(76) Inventors: Daniel Goldberger, Boulder, CO (US); Eric Shreve, Louisville, CO (US); Wayne Siebrecht, Golden, CO (US); Benny Pesach, Rosh Haayin (IL); Gidi Pesach, Kfar Vitkin (IL); Gabby Bitton, Jerusalem (IL); Ron Nagar, Tel Aviv (IL); Dalia Argaman, Hod-Hasharon (IL); Stephen Bellomo, Zircon Yacov (IL); Robert Larson, Perkasie, PA (US); Larry Johnson, Golden, CO (US); Jill Klomhaus, Niwot, CO (US)

Correspondence Address: PATENTMETRIX, 14252 CULVER DR. BOX 914, IRVINE, CA, 92604, US

(21) Appl. No.: 11/420,037

(22) Filed: May 24, 2006

(51) Int. Cl. C12Q 1/54; A61K 9/22

(52) U.S. Cl. 435/14; 604/890.1

(57) ABSTRACT

The present invention is directed toward a method of measuring a blood parameter and, in particular, a glucose measurement system controlled by a novel controller. In one embodiment, the system has a controller, a pump, flush solution in a first reservoir, IV solution in a second reservoir, a first valve, a second valve, and a plurality of tubing placing the pump, reservoirs, and valves in fluid communication with each other. The system operates by establishing a first state where the first state has the first valve open and second valve closed, closing the first valve, instructing the pump to extract fluid from tubing having IV solution therein, sensing a presence of blood at a first location in the tubing, measuring a blood parameter, instructing the pump to re-infuse the extracted fluid back into the tubing, opening the second valve where the second valve permits flush solution to flow from the first reservoir through tubing, instructing the pump to extract fluid from tubing having flush solution therein, closing the second valve, instructing the pump to re-infuse the extracted fluid having flush solution therein back into the tubing, and opening the first valve.

### FIELD OF THE INVENTION

The present invention relates generally to systems and methods for automatically measuring physiological parameters and blood constituents, and in particular, to a method and system for automated blood glucose measurement. In addition, the present invention relates to a controller for a physiological parameter and blood constituent measurement system. Further, the present invention relates to improved testing methods of such a controller.

### BACKGROUND OF THE INVENTION

Physiological monitors are used in the medical field to examine various physiological parameters of patients. These physiological monitors allow health and medical professionals, as well as individual users, to determine the current status of one or more physiological parameters and monitor these parameters over a period of time.

This information is extremely helpful in medical treatment. For example, a patient's blood chemistry comprises important diagnostic information that is critical to patient care. The measurement of blood analytes is often a critical prerequisite to determining the proper dosing and administration regimen of drugs. Blood analytes and parameters tend to change frequently, however, especially in the case of a patient under continual treatment, thus making the measurement process tedious, frequent, and difficult to manage.

Maintaining a consistent and normal blood glucose level is an arduous task as blood glucose level is prone to wide fluctuations, especially around mealtime. Controlling glucose levels requires the frequent measurement of blood glucose concentration in order to determine the proper amount and frequency of insulin injections or glucose dose. The ability to accurately measure analytes in blood, particularly glucose, is important in the management of patient's. Blood glucose levels must be maintained within a narrow range (about 3.5-6.5 mM). Glucose levels lower than this range (hypoglycemia) may lead to mental confusion, coma, or death. Sustained hyperglycemia (high glucose) has been linked to several of complications, including kidney damage, neural damage, and blindness.

Conventional glucose measurement techniques require puncturing a convenient part of the body (normally a fingertip) with a lancet, milking the finger to produce a drop of blood at the site, and depositing the drop of blood on a measurement device (such as an analysis strip). This lancing method is both painful and messy for the patient. The pain and inconvenience has additional and more serious implications of noncompliance.

Conventional Point-of-Care (POC) techniques for diagnostic blood testing are routinely performed manually at the bedside using a small sample of blood. SureStep® Technology, developed by Lifescan, is one example of a conventional Point-of-Care monitoring system. The SureStep® Technology in its basic form allows for simple, single button testing, quick results, blood sample confirmation, and test memory. In operation, the SureStep® Point-of-Care monitoring system employs three critical steps for performance. In a first step, the blood sample is applied to the test strip. The blood sample is deposited on an absorbent pad, which is touchable and promotes sample application. In addition, blood is retained and not transferred to other surfaces. The sample then flows one way through the porous pad to the reagent membrane, where the reaction occurs. The reagent membrane is employed to filter out red blood cells while allowing plasma to move through. In addition, it hides the red blood cells, thus simplifying glucose measurement.

In a second step, the glucose reacts with the reagents in the test strip. Glucose in the sample is oxidized by glucose oxidase (GO) in the presence of atmospheric oxygen, forming hydrogen peroxide (H₂O₂). H₂O₂ reacts with indicator dyes using horseradish peroxidase (HRP), forming a chromophore or light-absorbing dye. The intensity of color formed at the end of the reaction is proportional to the glucose present in the sample.

In a third step, the blood glucose concentration is measured with SureStep® meters. Reflectance photometry quantifies the intensity of the colored product generated by the enzymatic reaction. The colored product absorbs the light—the more glucose in a sample (and thus the more colored product on a test strip), the less reflected light. A detector captures the reflected light, converts it into an electronic signal, and translates it into a corresponding glucose concentration. The system is calibrated to give plasma glucose values.

In addition, prior art devices have conventionally focused upon manually obtaining blood samples from in-dwelling catheters. Such catheters may be placed in venous or arterial vessels, centrally or peripherally. For example, Edwards LifeSciences' VAMP Plus Blood Sampling System provides a method for the withdrawal of blood samples from pressure monitoring lines. The blood sampling system is designed for use with disposable and reusable pressure transducers and for connection to central line catheters, venous, and arterial catheters where the system can be flushed clear after sampling. The blood sampling system mentioned above, however, is for use only on patients requiring periodic withdrawal of blood samples from arterial and central line catheters that are attached to pressure monitoring lines.

The VAMP Plus design provides a manual needleless blood sampling system, employing a blunt cannula for drawing of blood samples. In addition, a self-sealing port reduces the risk of infection by stopcock contamination. The VAMP Plus system employs a large reservoir with two sample sites. Two methods may be used to draw a blood sample in the VAMP Plus Blood Sampling System. The first method, the syringe method for drawing blood samples, first requires that the VAMP Plus is prepared for drawing a blood sample by drawing a clearing volume. To draw a blood sample, it is recommended that a preassembled packaged VAMP NeedleLess cannula and syringe is used. Then, the syringe plunger should be depressed to the bottom of the syringe barrel. The cannula is then pushed into the sampling site. The blood sample is then drawn into the syringe. A blood transfer unit is then employed to transfer the blood sample from the syringe to the vacuum tubes.

The second method allows for a direct draw of blood samples. Again, the VAMP Plus is first prepared for drawing a blood sample by drawing a clearing volume. To draw a blood sample, the VAMP direct draw unit is employed. The cannula of the direct draw unit is pushed into the sampling site. The selected vacuum tube is inserted into the open end of the direct draw unit and the vacuum tube is filled to the desired volume.

The abovementioned prior art systems, however, have numerous disadvantages. In particular, manually obtaining blood samples from in-dwelling catheters tends to be cumbersome for the patient and healthcare providers.

In the light of above described disadvantages, there is a need for improved methods and systems that can provide comprehensive blood parameter testing. What is also needed is a programmable, automated system and method for obtaining blood samples for testing certain blood parameters and data management of measurement results, thus avoiding human recording errors and providing for central data analysis and monitoring. In addition, an improved mechanism of controlling blood parameter testing is needed.

### SUMMARY OF THE INVENTION

The present invention is directed toward a method of measuring a blood parameter and, in particular, a glucose measurement system controlled by a novel controller. In one embodiment, the present invention is directed toward a method for measuring a blood parameter using a system having a controller, a pump, flush solution in a first reservoir, IV solution in a second reservoir, a first valve, a second valve, and a plurality of tubing placing the pump, reservoirs, and valves in fluid communication with each other, comprising the steps of: a) establishing a first state wherein the first state has the first valve open and second valve closed, wherein the first open valve permits IV solution to flow from the second reservoir through tubing and to a patient; b) closing the first valve; c) instructing the pump to extract fluid from tubing having IV solution therein; d) sensing a presence of blood at a first location in the tubing; e) measuring a blood parameter; and f) instructing the pump to re-infuse the extracted fluid back into the tubing; g) opening the second valve where the second valve permits flush solution to flow from the first reservoir through tubing; h) instructing the pump to extract fluid from tubing having flush solution therein; i) closing the second valve; j) instructing the pump to re-infuse the extracted fluid having flush solution therein back into the tubing; and k) opening the first valve. The sensing of a presence of blood at a first location in the tubing is performed using a blood presence sensor.

Optionally, the method further comprises a third valve where the third valve is open during said first state and closed when the second valve is open. Optionally, the method further comprises a sample port, which is located between the blood presence sensor and the patient along the line of fluid flow between said patient and the blood presence sensor. Optionally, the sample port is used to access blood in order to measure a blood parameter, such as glucose, using an appropriate sensor, such as a glucose oxidase strip.

Optionally, the re-infusion of fluid back into tubing occurring after the measurement of a blood parameter occurs based upon a manual instruction from a user. Optionally, the re-infusion of fluid back into tubing occurring after the measurement of a blood parameter occurs automatically. Optionally, the automatic re-infusion is initiated based upon a signal from the blood presence sensor or pump. Optionally, the automatic re-infusion is initiated based upon a predefined period of time. Optionally, at least one of the valves is a pinch valve. Optionally, the pump is a syringe pump.

The controller controls the measurement of a blood parameter in a system having a pump, flush solution in a first reservoir, IV solution in a second reservoir, a first valve, a blood presence sensor, a second valve, a plurality of tubing placing the pump, reservoirs, and valves in fluid communication with each other, and a plurality of data connections placing the controller in data communication with the valves, blood presence sensor, and pump. The controller comprises a processor that a) issues instructions to establish a first state in the system, where the first state has the first valve open and second valve closed, wherein the first open valve permits IV solution to flow from the second reservoir through tubing and to a patient; b) instructs the first valve to close; c) instructs the pump to extract fluid from tubing having IV solution therein; d) receives a signal from the blood presence sensor indicating the presence of blood at a first location in the tubing; f) instructs the pump to re-infuse the extracted fluid back into the tubing; g) instructs the second valve to open wherein the second valve permits flush solution to flow from the first reservoir through tubing; h) instructs the pump to extract fluid from tubing having flush solution therein; i) instructs the second valve to close; j) instructs the pump to re-infuse the extracted fluid having flush solution therein back into the tubing; and k) instructs the first valve to open.

Optionally, the processor is in data communication with a third valve which is open during the first state and closed when the second valve is open. Optionally, the processor is in data communication with a sample port. Optionally, the sample port is used to access blood in order to measure a blood parameter, such as glucose. Optionally, the processor instructs the pump to re-infuse fluid back into said tubing based upon a signal from a pump or blood presence sensor.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 depicts a block diagram of an embodiment of the automated blood parameter testing apparatus of the present invention;

FIG. 2 is a schematic diagram of one embodiment of the automated blood parameter testing apparatus of the present invention;

FIG. 3 is a schematic diagram of a second embodiment of the automated blood parameter testing apparatus of the present invention;

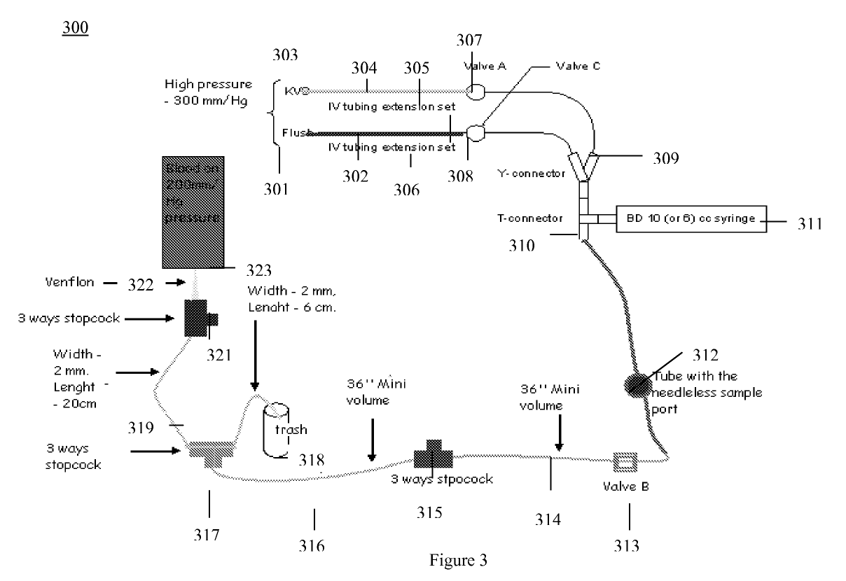

FIG. 4 is a schematic diagram of a third embodiment of the automated blood parameter testing apparatus of the present invention;

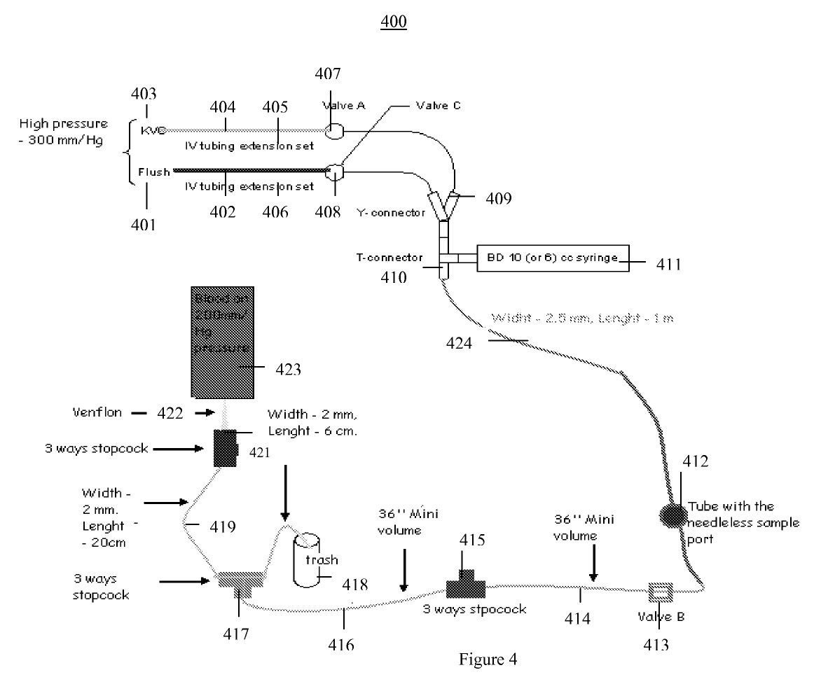

FIG. 5 illustrates a patient hemoglobin graph versus deviation from the sample obtained via automated blood parameter testing apparatus;

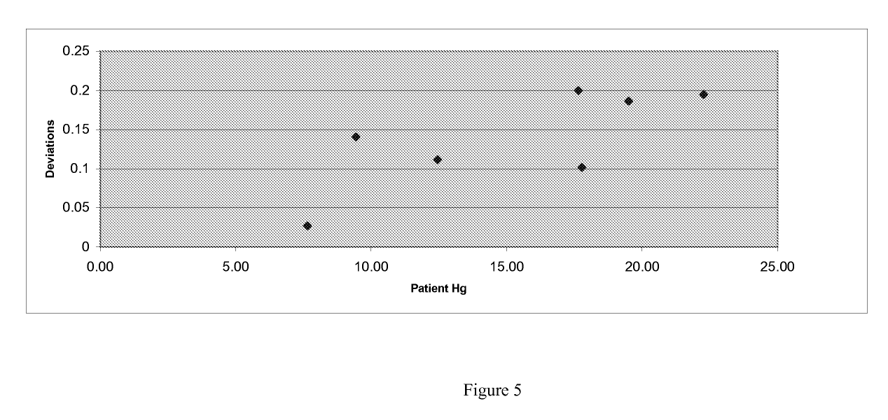

FIG. 6 is a blood sensor as used in the circuit of the automated blood parameter testing apparatus of the present invention;

FIG. 7 illustrates components of a monitor of the automated blood parameter analysis system of the present invention;

FIG. 8 depicts the components of a computing device as used in one embodiment of the automated blood parameter analysis system of the present invention; and

FIG. 9 depicts communication channels between a plurality of monitors with a central monitoring station in the blood parameter analysis system of the present invention.

### DETAILED DESCRIPTION

The present invention is directed to a controller to be used in a blood parameter testing system, such as the one described in co-pending U.S. patent application Ser. No. 11/386,078, incorporated herein by reference. Reference will now be made in detail to specific embodiments of the invention. While the invention will be described in conjunction with specific embodiments, it is not intended to limit the invention to one embodiment. Thus, the present invention is not intended to be limited to the embodiments described, but is to be accorded the broadest scope consistent with the disclosure set forth herein.

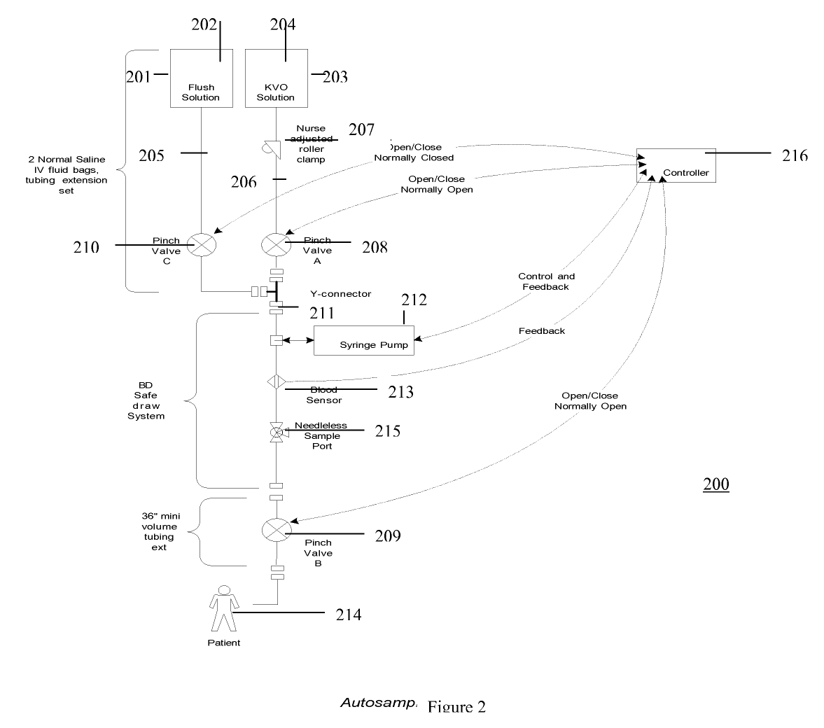

Referring to FIG. 2, a schematic diagram of one embodiment of the automated blood parameter testing apparatus of the present invention is depicted. The automated blood parameter testing apparatus 200 of the present invention comprises a reservoir 201 containing flush solution 202, a reservoir 203 containing IV solution (hereinafter, “KVO”) 204, distal tubes 205, 206 originating from the flush reservoir 201 and the KVO reservoir 203, a roller clamp 207, a valve A 208, a valve B 209, a valve C 210, a Y connector 211 connecting the tubing originating from the valve A 208 and valve C 210, a syringe pump 212 to move fluid bidirectionally in the tubing and a blood presence sensor 213 to determine the presence of blood, hemoglobin concentration, oxygenation status of blood, the concentration of blood analytes and/or the concentration of blood glucose.

The pinch valve B 209 forms the interface between the patient 214 and a sample port 215. In one embodiment, the tubing between the patient 214 and the sample port 215 is 36″ mini volume tubing, however other tubing of appropriate size can be used. The valves can be any type of valve, including a pinch valve or stopcock.

In one embodiment, blood presence sensor 213 is used for determining the presence of blood in the tube for analysis. In another embodiment, the sensor 213 is used for determining the presence of undiluted blood in the tube for analysis. Optionally, the sensor 213 is used to verify that no bubbles are present in the fluid contained in the tube. In an alternative embodiment, the sensor 213 is used to determine the oxygenation level of the blood and uses the oxygenation level to calibrate or adjust the glucose calculation.

The operation of each of the aforementioned valves, pumps, and sensors are controlled by controller 216. Controller 216 comprises at least one processor operating software that is capable of conducting tests on the system 200 and conducting blood parameter testing in accordance with user commands and/or predefined testing protocols. The controller 216 used in the present invention can be any appropriate microcontroller, control device, control circuit or any suitable computing device including, but not limited to laptop, personal digital assistant (PDA), mobile phones, wireless activated gadgets, infrared devices and bluetooth devices. It should be appreciated that the controller 216 is in data communication, either through wired or wireless connections, with the valves, pump, blood presence sensor, and/or sample port. It should also be appreciated that when actions are described in the present system, such as beginning in a first state, opening and closing valves, causing a pump to infuse or re-infuse fluid, such actions occur based upon instructions being issued from at least one processor in the controller and transmitted to the system component through wired or wireless data communication.

In one embodiment, the controller operates by beginning in a first state, e.g. a non-measurement state. In this first state, the pinch valve A 208 and the pinch valve B 209 are open while the valve C 210 is closed. The KVO solution 204 is in fluid communication with the vascular access point of the patient 114. The KVO solution 204 is maintained at a slight positive pressure, usually by gravity, thus completely filling the tube with the solution.

A start process is activated in the controller 216. The start process can be activated using a plurality of predefined protocols or can be manually activated by a user. The predefined protocols can establish a plurality of times when a measurement must occur based upon any set of parameters, including but not limited to time, historical measurements, historical glucose measurements, other physiological measurements, such as blood pressure, pulse rates, and oxygenation level, eating schedule, the patient's sex, the patient's age, and/or the time since the last measurement.

When the start process is activated, the controller communicates a signal to close valve A. Once valve A is closed, the controller communicates a signal to the syringe pump 212 to extract fluid into the syringe body. Fluid flowing from tube 206 fills the reservoir of the pump mechanism, generating negative pressure in the tube. The negative pressure at the extreme end of the tubing results in the withdrawal of blood from the vascular access point 214. As blood enters the tube and moves upward, it eventually passes the blood presence sensor 213. The blood sensor activates and sends a signal to the controller 216 that blood is present in the section of tubing being monitored by the blood sensor 213.

With blood being present at the sensor 213, it is assured that blood is also present at point 215, the sample port. The sample port 215 can then be used by a healthcare provider to access blood in order to manually test it. In a preferred embodiment, an automated glucose monitoring device, as described in the aforementioned co-pending, co-assigned patent applications, can be used to automatically sample blood and measure glucose.

In one embodiment, the measurement element used in either manual or automatic measurement is a glucose oxidase strip or any other glucose measurement strip known to diabetes patients or persons of ordinary skill in the art. In another embodiment, measurement element is a fixed sensor.

The blood sample on the reagent strip reacts with the reagents in the reagent strip; thus, the resulting color change is read from the backside of the test strip via the optical sensor. The optical sensor signals are converted by electronic meter into a numerical readout on display, which reflects a numerical glucose level of the blood sample. Once the glucose concentration of the blood sample is displayed on the display panel, the data is transferred to the central monitoring station for future use.

Once a measurement reading is completed, the controller activates the syringe pump 212 to re-infuse the fluid back into the tubing which, in turn, causes the blood sample to re-infuse back to the patient 214. The controller 216 activates the re-infusion process based upon a manual instruction from a user or automatically. In an automated implementation, the automated glucose monitoring device, located at point 215, communicates a measurement completion signal and/or measurement data to the controller 216, thereby signaling the controller 216 to initiate the re-infusion process. Once the re-infusion process is complete, as determined by a feedback signal from the syringe pump 212 or the blood presence sensor 213 or by a pre-defined period of time, the controller 216 opens the valve C 210 and the flush tubing system of the automated blood parameter testing apparatus is activated.

The flush solution 202 is maintained at a slight positive pressure by gravity. Therefore, the moment valve C 210 is opened, the flush solution 202 flows into the tubular system. Controller 216 closes valve 209 and activates the syringe pump 212 again and the plunger is extracted, thus filling the reservoir of the pump mechanism with the flush solution 202. Once the extraction is complete and a signal is transmitted to controller 216, controller 216 closes valve C 210 and opens valve B 209. Controller then communicates a signal to syringe pump 212 to re-infuse the fluid into the tubing, including the 36″ mini volume tubing, between the pinch valve B 208 and the distal end. The automated blood parameter testing apparatus is now again ready for the next sample analysis and flushing.

In an alternative embodiment; additional stopcock 317 and waste container 318 are added to the system. With these additional elements; blood withdrawn from the patient and/or flush solution may be diverted to the waste container as required instead of being returned to the patient's vascular system.

In an alternative embodiment; additional extension tubing 424 is added to the system as shown in order to reduce or eliminate sample dilution. The extension tube 424 is preferably in the range of length 0.5 to 2 m, more preferably 1 m, and width 2.5 mm.

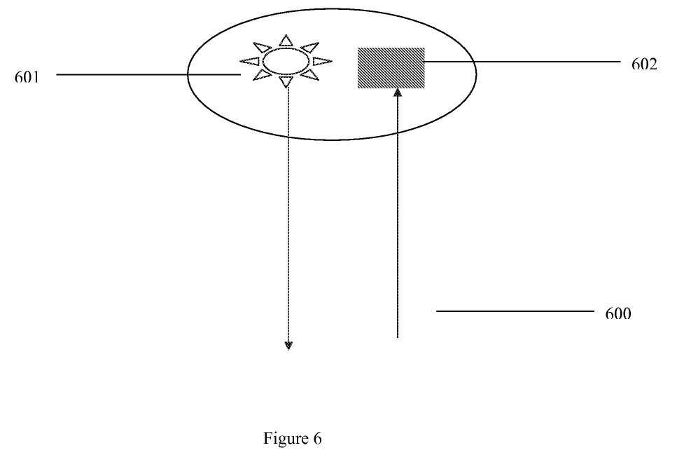

Referring now to FIG. 6, a blood presence sensor as used in the circuit of the automated blood parameter testing apparatus of the present invention is depicted. The sensor is used for monitoring the presence or absence of blood in the circuit to enhance the reliability of the automated blood parameter testing apparatus of the present invention. Although in a preferred embodiment the blood sensor is used for the detection of the presence or absence of blood in the circuit, it is not limited to such use. The sensor is also employed to detect the dilution of blood or detect other blood parameters, such as but not limited to, oxygenation or hemoglobin concentration, which are subsequently useful in improving the accuracy of the glucose determination.

Blood sensor 600 comprises an illumination source 601 and a detector 602. Illumination source 601 is used to trans-illuminate the tubing. The illumination source can be a single, multi-wavelength laser diode, a tunable laser or a series of discrete LEDs or laser diode elements, each emitting a distinct wavelength of light selected from the near infrared region. Alternatively, the illumination source can be a broadband near infrared (IR) emitter, emitting wavelengths as part of a broadband interrogation burst of IR light or radiation, such as lamps used for spectroscopy.

At least one detector 603 detects light reflected and/or transmitted by sample blood. The wavelength selection can be performed by either sequencing single wavelength light sources or by wavelength selective elements, such as using different filters for the different detectors or using a grating that directs the different wavelengths to the different detectors. The detector converts the reflected light into electrical signals indicative of the degree of absorption light at each wavelength and transfers the converted signals to an absorption ratio analyzer such as a microprocessor. The analyzer processes the electrical signals and derives an absorption (e.g., a reflection and/or transmittance) ratio for at least one wavelength. The analyzer then compares the calculated ratio with predetermined values to detect the concentration and/or presence of an analyte such as, but not limited to glucose, hematocrit levels and/or hemoglobin oxygenation levels in the patient blood sample. For example, changes in the ratios can be correlated with the specific near infrared (IR) absorption peak for glucose at about 1650 nm or 2000-2500 nm or around 10 micron, which varies with concentration of the blood analyte. Alternatively, the algorithm detects rate of change or temporal pattern to determine the concentration and/or presence of an analyte.

In one embodiment, the abovementioned blood sensor 600 is an electrochemical sensor capable of detecting the presence of, and enabling the measurement of, the level of an analyte in a blood sample via electrochemical oxidation and reduction reactions at the sensor. In another embodiment, the sensor 600 is an optochemical sensor capable of detecting the presence of and enabling the measurement of the level of an analyte in a blood or plasma.

In one embodiment, blood sensor 600 establishes the presence of blood in the tube and subsequently activates other components of the blood parameter testing apparatus, such as advancement of a glucose oxidase strip and measurement by the electronic meter, for further analysis of the blood sample. Blood sensor 600 also determines whether the blood available in the tube is undiluted and bubble-free in the fluid circuit.

As described above, the method of detecting whether undiluted blood has reached the proximity of the sensor and is ready for sampling is to illuminate the tubing in the proximity of the sensor. Based upon the transmitted and/or reflected signal, the device can establish whether the fluid in the specific segment is undiluted blood. Dead space is managed by actively sensing the arrival and departure of blood from the sensor location.

In addition, blood sensor 600 is capable of other blood analysis functions, including but not limited to, determining the oxygenation level of the blood and using the oxygen status to adjust or calibrate the glucose calculation. In one embodiment, the optically measured hematocrit level is used to correct for the influence of hemodilution on blood analytes such as, but not limited to, glucose. Hematocrit levels and hemoglobin oxygenation levels are accurately measured using one or more wavelengths. Other combinations regarding the number and type of optical wavelengths and the parameters to be corrected can be used according to known correlations between blood parameters.

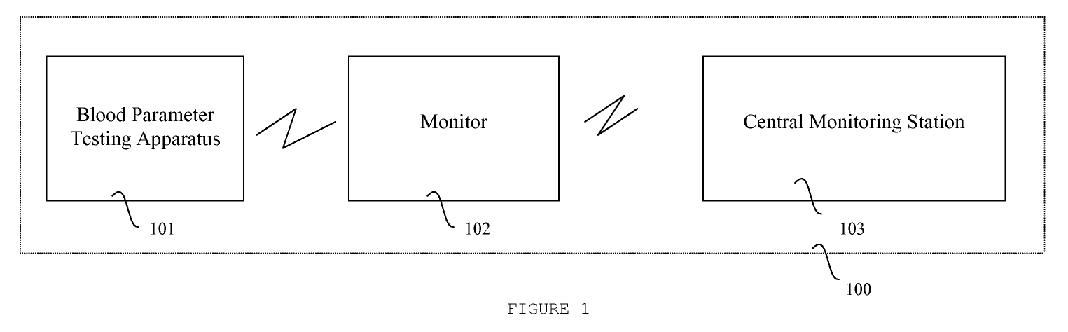

As described with reference to FIG. 1 above, in one embodiment, the blood parameter testing apparatus of the present invention is set up to communicate with patient monitors and/or central stations and/or the Internet. Once the blood glucose level of the patient is ascertained, the processed data from the glucose meter is stored in the local memory of the glucose meter and subsequently transmitted to a monitor. In one embodiment, the data stored within the glucose meter is preferably transferred to the monitor through appropriate communication links and an associated data modem. In an alternative embodiment, data stored within the glucose meter may be directly downloaded into the monitor through an appropriate interface cable.

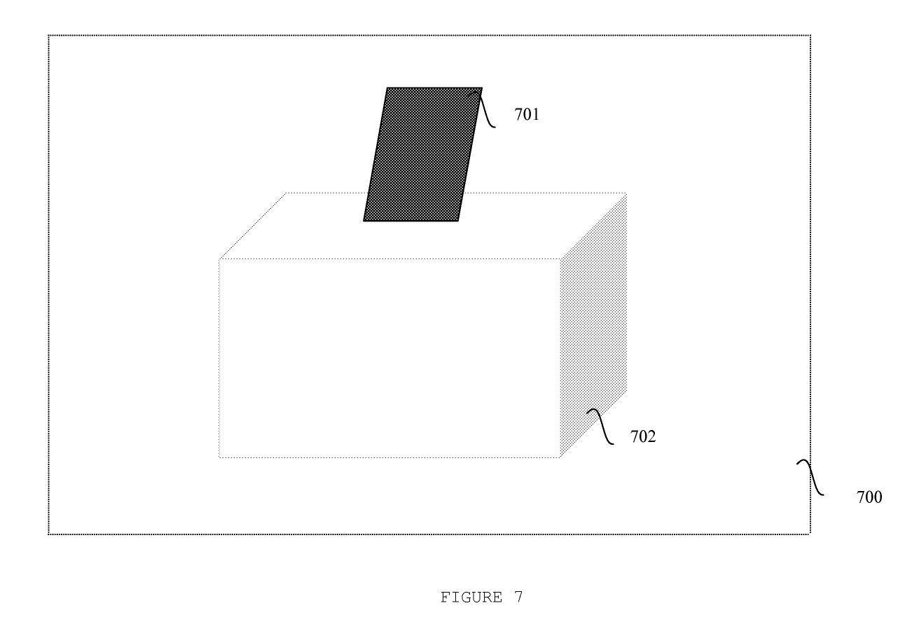

Referring now to FIG. 7, the components of the monitor as used in the automated blood parameter measuring system of the present invention is depicted. Monitor 700 comprises a glucose meter 701 and a computing device 702, which are preferably portable. Computing device 702 may be, but is not limited to, a portable computer such as personal digital assistant (PDAs), electronic notebook, pager, watch, cellular telephone and electronic organizer. Glucose meter 701 is connected to or docked with computing device 702 to form an integral unit. Glucose meter 701 may be inserted into an access slot (not shown) in computing device 702, may grip its housing, or interconnect in any other suitable manner as is well known to those of skill in the art. When glucose meter 701 is docked with computing device 702, computing device 702 identifies the card 701 and loads the required software either from its own memory or from the card.

Thus, glucose meter 701 includes the software necessary to process, analyze and interpret the recorded diabetes patient data and generate an appropriate data interpretation output. The results of the data analysis and interpretation performed upon the stored patient data by the monitor 700 are displayed in the form of a paper report generated through a printer (not shown) associated with the monitor 700. Alternatively, the results of the data interpretation procedure may be directly displayed on a graphical user interface unit (not shown) associated with the central monitoring station (not shown).

The software uses a blend of symbolic and numerical methods to analyze the data, detect clinical implications contained in the data and present the pertinent information in the form of a graphics-based data interpretation report. The symbolic methods used by the software encode the logical methodology used by healthcare providers as they examine patient logs for clinically significant findings, while the numeric or statistical methods test the patient data for evidence to support a hypothesis posited by the symbolic methods, which may be of assistance to a reviewing physician.

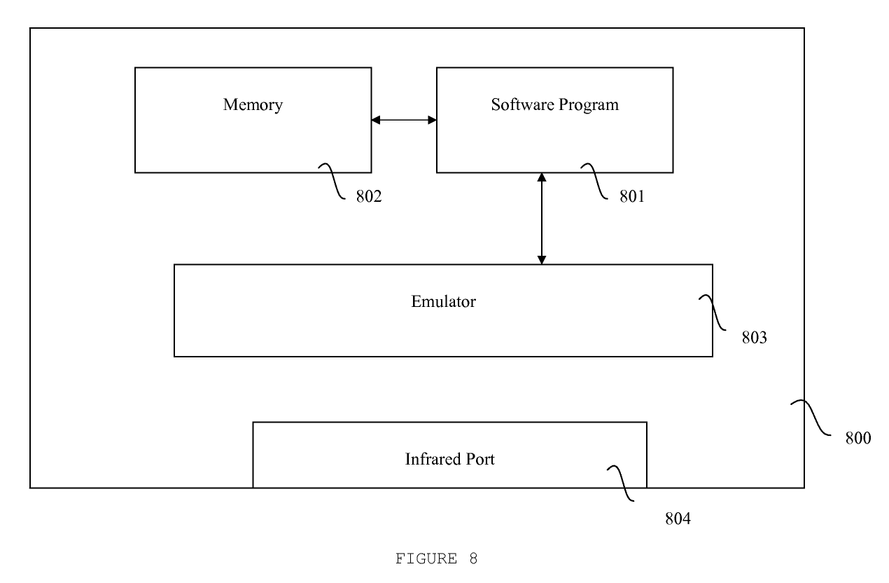

Referring to FIG. 8, the diagram depicts the components of a computing device as used in the blood parameter analysis system of the present invention. Computing device 800 preferably comprises software program 801, memory 802, emulator 803, and infrared port 804. Upon user request the information from the central monitoring station is received by software program 801 and stored in memory 802.

Software program 801 allows the user to perform queries on the stored information. For example, the user may wish to view a selected group of patients or all patients under observation. The user may set an alarm, when a desired sensor is in operation. The results of the user's query are displayed through a graphical user interface (GUI) on a display panel (not shown).

Operationally, a user may choose a person to be examined by selecting the appropriate glucose meter unit attached to that individual, using the GUI application. Each glucose meter consists of a unique identification. The selection causes the emulator, which emulates a remote control, to send instructions for that particular glucose meter. The instructions are sent via an infrared signal transmitted from the infrared port of the monitor to the photodetector (not shown) of the glucose meter, which is further conveyed to the sensor unit. The sensor unit is now initiated to communicate with the monitor. The monitor then receives the physiological signals from sensor unit and measures the desired physiological parameter.

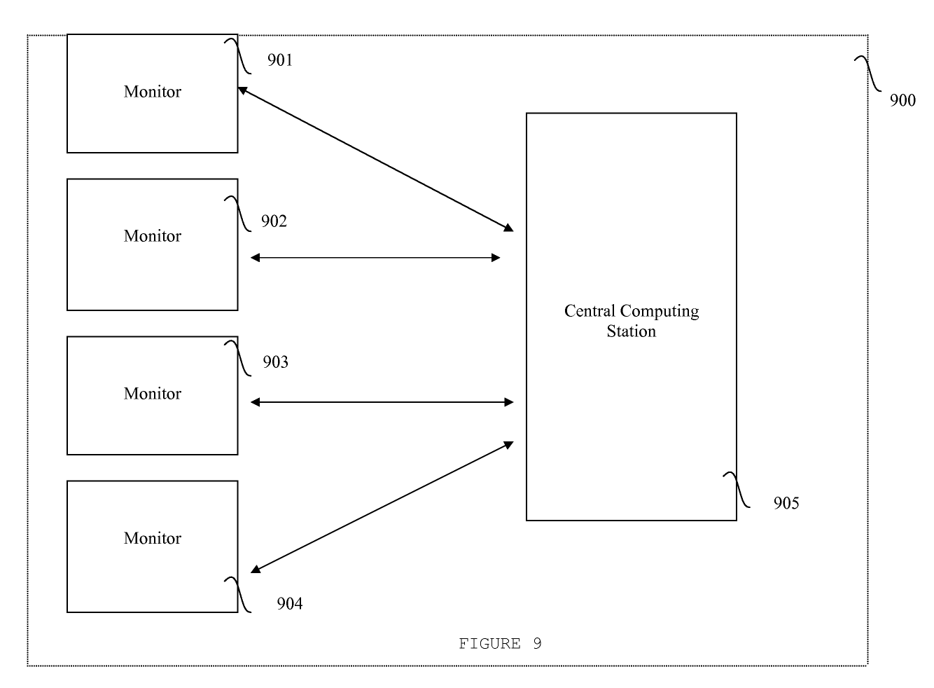

Referring now to FIGS. 1 and 9, the diagrams depict a communication scheme between a blood parameter testing apparatus 101, a plurality of monitors 102, 901, 902, 903, and 904 and a central monitoring station 103, 905. Monitors 102, 901, 902, 903, and 904 wirelessly transmit vital patient information, including but not limited to the measured blood glucose level to central monitoring station 103, 905. Medical conditions of a plurality of individual patients can be monitored from central monitoring station 905. An online database of the patients can be easily transported using a suitable relational database management system and an appropriate application programming language to the web server to make patient health conditions available on the World Wide Web.

In an alternative embodiment, either single or multiple lumen tubing structures may be attached to the catheter attached to the vascular access point. The tubing structure may vary depending upon functional and structural requirements of the system and are not limited to the embodiments described herein.

The automated system for periodically measuring blood analytes and blood parameters further includes alerts and integrated test systems. The alerts may also include alerts for a hemoglobin level below a defined level. In addition, the alerts may include alerts for hyperglycemia and hypoglycemia. The alerts may also include alerts for detection of air in a line and detection of a blocked tube.

Optionally, the control unit of the automated system for periodically measuring blood analytes and blood parameters enables input of user-defined ranges for blood parameters. Still optionally, the system alerts the user when the blood measurement falls outside of the user-defined ranges for blood parameters. The system may optionally alert the user when the rate of change of a parameter (eg blood glucose) is rising or falling too quickly or too slowly. Still optionally, the data from the system is correlated with other blood parameters to indicate an overall patient condition.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

We claim:

1. A method for measuring a blood parameter using a system having a controller, a pump, flush solution in a first reservoir, IV solution in a second reservoir, a first valve, a second valve, and a plurality of tubing placing said pump, said reservoirs, and said valves in fluid communication with each other, comprising the steps of:
a. establishing a first state wherein said first state has said first valve open and said second valve closed, wherein said first open valve permits IV solution to flow from said second reservoir through tubing and to a patient;
b. closing said first valve;
c. instructing said pump to extract fluid from tubing having IV solution therein;
d. sensing a presence of blood at a first location in said tubing;
e. measuring a blood parameter;
f. instructing said pump to re-infuse the extracted fluid back into said tubing;
g. opening said second valve wherein said second valve permits flush solution to flow from said first reservoir through tubing;
h. instructing said pump to extract fluid from tubing having flush solution therein;
i. closing said second valve;
j. instructing said pump to re-infuse the extracted fluid having flush solution therein back into said tubing; and
k. opening said first valve.

2. The method of claim 1 further comprising a third valve.

3. The method of claim 2 wherein said third valve is open during said first state.

4. The method of claim 2 wherein said third valve is closed when said second valve is open.

5. The method of claim 1 further comprising a sample port.

6. The method of claim 5 wherein the sensing of a presence of blood at a first location in said tubing is performed using a blood presence sensor.

7. The method of claim 6 wherein the sample port is located between said blood presence sensor and said patient along the line of fluid flow between said patient and the blood presence sensor.

8. The method of claim 7 wherein the sample port is used to access blood in order to measure a blood parameter.

9. The method of claim 8 wherein the blood parameter is glucose.

10. The method of claim 9 wherein the glucose is measured using a glucose oxidase strip.

11. The method of claim 1 wherein re-infusion of fluid back into said tubing occurring after the measurement of a blood parameter occurs based upon a manual instruction from a user.

12. The method of claim 1 wherein re-infusion of fluid back into said tubing occurring after the measurement of a blood parameter occurs automatically.

13. The method of claim 12 wherein the sensing of a presence of blood at a first location in said tubing is performed using a blood presence sensor.

14. The method of claim 13 wherein said automatic re-infusion is initiated based upon a signal from said blood presence sensor.

15. The method of claim 12 wherein said automatic re-infusion is initiated based upon a signal from said pump.

16. The method of claim 12 wherein said automatic re-infusion is initiated based upon a predefined period of time.

17. The method of claim 1 wherein at least one of said valves is a pinch valve.

18. The method of claim 1 wherein said pump is a syringe pump.

19. A controller for controlling the measurement of a blood parameter in a system having a pump, flush solution in a first reservoir, IV solution in a second reservoir, a first valve, a blood presence sensor, a second valve, a plurality of tubing placing said pump, said reservoirs, and said valves in fluid communication with each other, and a plurality of data connections placing said controller in data communication with said valves, blood presence sensor, and said pump, comprising a processor wherein said processor:
a. issues instructions to establish a first state in the system, wherein said first state has said first valve open and said second valve closed, wherein said first open valve permits IV solution to flow from said second reservoir through tubing and to a patient;
b. instructs said first valve to close;
c. instructs said pump to extract fluid from tubing having IV solution therein;
d. receives a signal from the blood presence sensor indicating the presence of blood at a first location in said tubing;
e. instructs said pump to re-infuse the extracted fluid back into said tubing;
f. instructs said second valve to open wherein said second valve permits flush solution to flow from said first reservoir through tubing;
g. instructs said pump to extract fluid from tubing having flush solution therein;
h. instructs said second valve to close;
i. instructs said pump to re-infuse the extracted fluid having flush solution therein back into said tubing; and
j. instructs said first valve to open.

20. The controller of claim 19 wherein said processor is in data communication with a third valve.

21. The controller of claim 20 wherein said processor instructs said third valve to be open during said first state.

22. The controller of claim 20 wherein said processor instructs said a third valve to be closed when said second valve is open.

23. The controller of claim 19 wherein said processor is in data communication with a sample port.

24. The controller of claim 23 wherein the sample port is used to access blood in order to measure a blood parameter.

25. The controller of claim 24 wherein the blood parameter is glucose.

26. The controller of claim 25 wherein the glucose is measured using a glucose oxidase strip.

27. The controller of claim 19 wherein the processor instructs the pump to re-infuse fluid back into said tubing based upon a manual instruction from a user.

28. The controller of claim 19 wherein the processor instructs the pump to re-infuse fluid back into said tubing based upon a signal.

29. The controller of claim 28 wherein said signal is from said blood presence sensor.

30. The controller of claim 28 wherein said signal is from said pump.
